(19) United States
(12) Patent Application Publication
(10) Pub. No.: US 2003/0175302 A1
(43) Pub. Date: Sep. 18, 2003

(54) Avian pneumovirus vaccine

(76) Inventor: Sagar M. Goyal, Roseville, MN (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/098,934

(22) Filed: Mar. 13, 2002

(51) Int. Cl. C12Q 1/68; A61K 39/12; A61K 39/155; C12N 7/00; C12N 7/01; C12N 7/04; C07K 1/00

(52) U.S. Cl. 424/211.1; 424/204.1; 435/6; 435/235.1; 435/236; 435/269

(57) ABSTRACT

Compositions and methods for ameliorating the clinical signs of an avian pneumovirus (APV) infection in a bird are disclosed. The compositions include immunologically effective amounts of an attenuated, cold-adapted APV. Methods for preparing an attenuated, cold-adapted APV composition are also described.

### TECHNICAL FIELD

This invention relates to avian vaccines, and more particularly to avian vaccines derived from avian pneumoviruses.

### BACKGROUND

Avian pneumovirus (APV) is a member of the Paramyxoviridae family of viruses. Pringle, Arch. Virol., 141:2251-2256 (1996). It is the etiological agent of turkey rhinotracheitis, causing an acute upper respiratory tract infection characterized by coughing, nasal discharge, tracheal rates, foamy conjunctivitis and sinusitis in young poults. In laying birds, there is also transient drop in egg production along with mild respiratory tract illness. Jones, Avian Pathol., 25:639-648 (1996). While uncomplicated cases of APV infection usually result in low mortality, secondary bacterial and/or viral infections can result in up to 25% mortality. Id.

APV was first detected in South Africa in 1978, and later diagnosed in the UK, France, Spain, Germany, Italy, Netherlands, Israel, and Asia. See Alexander, In Diseases of Poultry; 10^{th} Edition, Barnes et al., (eds.), 541-569 (1997); and Jones, supra. The first United States APV case was in Colorado in 1996. See Kleven, Proc. U.S. Animal Health. Assoc. 101^{st} Annual Mtg., 486-491 (1997). Subsequent APV infections were reported in Minnesota and neighboring states. See Lauer, Minnesota Poultry Testing Laboratory Monthly Report, (1999). By 1999, at least 37% of the turkey flocks in Minnesota were positive for APV antibodies, causing economic losses of approximately 15 million dollars per year.

The tremendous economic pressure caused by APV outbreaks has caused some farmers to expose young turkey flocks to homogenized lungs obtained from APV infected turkeys in a desperate attempt to immunize young poults. These drastic steps taken by farmers are not safe and effective methods for protecting turkeys from APV infection. Thus, there exists a need for safe and effective vaccines against APV infections in birds including turkeys.

### SUMMARY

In one aspect, the invention features a composition that includes an immunologically effective amount of an attenuated, avian pneumovirus (APV). In one embodiment, the attenuated APV is sequestered. In some embodiments, the composition further includes an acceptable pharmaceutical carrier. In other embodiments, the attenuated APV is cold-adapted to grow at 31° C.

These compositions containing an immunologically effective amount of an attenuated APV are effective for lowering the risk of an APV infection in wild birds and domesticated birds. In particular, the compositions are useful for preventing an APV infection in poultry including turkeys, chickens, ducks, geese, pheasants, partridges, guinea fowl, and peacocks. In addition, the APV compositions are effective for ameliorating the clinical signs of an APV infection in a challenged bird.

In another aspect, the inventions feature methods for preparing an attenuated APV composition by infecting or inoculating a cell culture with an APV, and serially propagating the infected cell culture. If a cold-adapted, attenuated APV composition is desired, the infected cell culture is propagated at successively lower temperatures until the APV becomes both attenuated and cold-adapted. In some embodiments, methods for preparing an attenuated, cold-adapted APV further include the step of removing the attenuated, cold-adapted APV from the infected as cell culture. The cell cultures can be avian or non-avian cell cultures, or a combination thereof in any order. For example, cell cultures can include Vero cells, quail tumor cells (QT-35 cells) or chicken embryo fibroblasts (CEF). In some embodiments, the APV is selected from the group consisting of the European A, European B, Colorado, Minnesota 1A, Minnesota 1B, Minnesota 2A, and Minnesota 2B isolates.

In some embodiments, the infected cell culture is serially passaged at least 20 times, at least 40 times, at least 60 times, or at least 100 times, or any number of passages between 10 and 110 passages. For example, in one embodiment the infected cell culture is serially passaged 41 times. In another embodiment, the infected cell culture is serially passaged 65 times. Lowering the temperature at which the cell culture is grown can be done at any stage during serial passaging. For example, the temperature can be lowered between passage number 40 and passage number 60, between passage number 60 and passage number 80, or between passage number 50 and passage number 75. For example, the temperature can be lowered every 5 passages, every 8 passages, or every 10 passages. Additionally, the number of degrees the temperature is lowered can vary, depending on the cells in culture and the passage number. For example, the temperature can be successively lowered by 2° C., by 5° C., or by 10° C. In one embodiment, an attenuated, cold-adapted APV of the invention is passaged 41 times at 37° C., 8 times at 35° C., 8 times at 33° C., and 8 times at 31° C.

These methods produce an attenuated APV that is effective for reducing or preventing the incidence of the clinical signs of an APV infection in poultry and, in particular, turkeys and chickens. Attenuated, cold-adapted APV produced by methods of the invention also are effective for reducing or preventing the incidence of the clinical signs of an APV infection in poultry.

In another aspect, the invention features a method for preparing an attenuated APV composition that includes the steps of inoculating or infecting an avian cell culture with an APV, propagating the APV in the avian cell culture, inoculating or infecting a non-avian cell culture with an APV isolated from the propagated avian cell culture, propagating the non-avian infected cell culture until the APV becomes attenuated, and isolating the attenuated APV from the non-avian infected cell culture. A method for preparing an attenuated, cold-adapted APV composition also is featured, wherein the non-avian cell culture is propagated at successively lower temperatures until the APV becomes attenuated and cold-adapted.

In another aspect, the invention features a method for reducing the risk of an APV infection in a bird by inoculating a bird with an immunologically effective amount of an attenuated APV composition. Such a composition can be an attenuated, cold-adapted APV In some embodiments, the inoculated bird is allowed to become seropositive. Although many different dosages may be used, particularly useful dosages include inoculating a bird with at least 1.0×10¹ tissue culture infective dose (TCID₅₀) of the attenuated APV composition, at least 1.0×10² TCID₅₀ of the attenuated APV composition, at least 1.0×10³ TCID₅₀ of the attenuated APV composition, at least 1.0×10⁵ TCID₅₀ of the attenuated, cold-adapted APV composition, or at least 1.0×10⁷ TCID₅₀ of the attenuated APV composition. Similar dosages can be used of the attenuated, cold-adapted APV composition.

Any method of inoculation can be used including applying the composition to one or more eyes of a bird and/or one or more nostril of a bird, aerosolizing the vaccine into the turkeys' habitat, or supplying the attenuated or the attenuated, cold-adapted APV composition in the drinking water of a bird. Inoculated birds can be members of a flock of birds and the inoculated or vaccinated birds can cause a majority of the flock to become seropositive. Alternatively, all birds may be vaccinated simultaneously be aerosolization, by placing the vaccine in drinking water, or by instilling the vaccine in the eye and/or nostril of all birds. In some embodiments, the method is effective for reducing the incidence of the clinical signs of an APV infection in a challenged bird.

In another aspect, the invention features an inoculated bird, which is a bird containing an inoculant of an immunologically effective amount of an attenuated APV or an attenuated, cold-adapted APV. In some embodiments, the bird is allowed to become or is seropositive for APV. In another aspect, the invention features a body part, such as a meat portion, of an inoculated or vaccinated bird. In particular, these birds can be turkeys.

In yet another aspect, the invention features compositions containing immunologically effective amounts of an attenuated, cold-adapted, APV that has been inactivated. In some embodiments, the composition further includes an acceptable pharmaceutical carrier or adjuvant. Compositions containing an inactivated, attenuated, cold-adapted APV are also effective for lowering the risk of an APV infection in poultry, such as chickens or turkeys, and in other domesticated and wild birds. The compositions are also effective for ameliorating the clinical signs of an APV infection in a challenged bird. In some embodiments, inactivation is a formalin or β-propiolactone inactivation.

In another embodiment, the invention features a composition containing an immunologically effective amount of an attenuated APV that became attenuated by serially propagating an APV in a non-avian host, such as a Vero cell. An attenuated, cold-adapted APV can be generated by propagating the APV in a non-avian host at successively lower temperatures. In other embodiments, the composition containing an immunologically effective amount of an attenuated APV or an attenuated, cold-adapted APV was serially propagated in an avian host before being propagated in a non-avian host in vitro. Successive reductions in temperature can be performed on the avian host or the non-avian host to produce an attenuated, cold-adapted APV.

In another embodiment, a method for preparing an attenuated APV composition includes the steps of infecting a host with an APV, propagating the APV in the host, infecting a cell culture with an APV isolated from the propagated host, propagating the infected cell culture until the APV becomes attenuated, and perhaps isolating the attenuated, cold-adapted APV from the non-avian infected cell culture. An APV can be attenuated and made cold-adapted by propagating the infected cell culture at successively lower temperatures. Useful hosts for such a method include embryonated chicken eggs, embryonated turkey eggs, and tracheal organ cultures. Useful cell cultures for such a method include chicken embryo fibroblast, quail tumor cell lines, and Vero cells.

In another aspect, the invention features an article of manufacture containing an attenuated APV or an attenuated, cold-adapted APV composition. The compositions can be combined with packaging materials and instructions for their use. The articles of manufacture may combine one or more attenuated and/or cold-adapted APV vaccines. In addition, the articles of manufacture of the invention may further include antibodies, indicator molecules, and/or other useful agents for detecting other avian diseases. The instructions can describe how an attenuated or an attenuated, cold-adapted APV vaccine is effective for preventing the incidence of an APV infection, preventing the occurrence of the clinical signs of an APV infection, ameliorating the clinical signs of an APV infection, lowering the risk of the clinical signs of an APV infection, lowering the occurrence of the clinical signs of an APV infection and/or lowering the spread of APV infections in birds.

Articles of manufacture can also include diagnostic molecules that are effective for detecting the presence of APV or other avian infections in birds. Moreover, it is to be ago understood that attenuated APV or attenuated, cold-adapted APV vaccines themselves and/or articles of manufacture that include attenuated APV or attenuated, cold-adapted APV vaccines can include other components conventional to the art, for example sterile water, pharmaceutical carriers, vaccine carriers, and buffers that are useful for maintaining the viability of the APV vaccines. The APV vaccines and/or articles of manufacture may also contain other attenuated and/or cold-adapted virus strains, microorganisms, and antigens that protect the inoculated birds against other avian diseases. Conveniently, the attenuated or attenuated, cold-adapted APV vaccines of the invention may be provided in a pre-packaged form in quantities sufficient for a protective dose for a single bird or for a pre-specified number of birds in, for example, sealed ampoules, capsules or cartridges.

It is to be further understood that each of the embodiments may be combined with any of the other embodiments described herein. For example, the methods disclosed herein are useful with any of the compositions, or articles of manufacture, and the dosages may be used in any of the various embodiments described herein.

Advantages of the invention include a safe and effective way to protect birds, especially turkeys, from an APV infection. Moreover, the invention can provide methods for raising antibodies to APV to be used in diagnostic kits and may be used in the diagnostic kits for detecting the presence of APV.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### DETAILED DESCRIPTION

The invention features methods, compositions of matter, and articles of manufacture (kits) containing an avian composition or vaccine that is derived from an avian pneumovirus (APV). An avian composition or vaccine can contain an immunologically effective dosage of an attenuated, cold-adapted APV. Such a vaccine can be produced by serially propagating a virulent form of APV in a cell culture at successively lower temperatures until the APV becomes attenuated and cold-adapted. These APV vaccines are both safe and immunogenic when administered to birds. Vaccinated birds are seropositive for anti-APV antibodies and resistant to infection by or challenge with a virulent APV. Using the compositions and/or vaccines described herein is an effective way for preventing, ameliorating, lowering the risk of, lowering the occurrence of and/or spread of APV infections in birds. An avian composition or vaccine of the invention can contain an immunologically effective dosage of an inactivated, attenuated, cold-adapted APV.

Compositions Containing Attenuated, Cold-Adapted Vaccines and Their Use

As used herein, live vaccines are synonymous with attenuated vaccines. Attenuated, cold-adapted APV vaccines are compositions containing a sufficiently attenuated APV that is adapted to grow at lower-than-normal temperatures. Vaccines of the invention are useful for any type of bird susceptible to APV infection including domesticated and wild birds. In particular, the vaccines described herein are useful for inoculating and/or treating birds living in flocks or other types of close living arrangements where an APV infection can rapidly spread from bird to bird. Domesticated birds that may benefit from receiving an APV vaccine include poultry such as turkeys, chickens, ducks, geese, pheasants, partridges, guinea fowl, peacocks, and any other type of domesticated bird. Wild birds that may benefit from receiving an APV vaccine include starlings, sparrows, turkeys, ducks, geese, pheasants, partridges, guinea fowl, peacocks, and any other type of wild bird that may contract an APV infection and/or transmit APV infection to a domesticated bird.

An immunologically effective dosage of an attenuated, cold-adapted APV vaccine is a dosage that, when administered to a bird, elicits an immunological response in the bird but does not cause the bird to develop severe clinical signs of an APV infection. A bird that has received an immunologically effective dosage is an inoculated bird or a bird containing an inoculant of an immunologically effective amount of an isolated attenuated, cold-adapted APV. When the bird elicits an immunological response it is considered seropositive, i.e., the bird produces a detectable amount of anti-APV antibodies. Methods for detecting an immunological response in a bird are known, e.g., Chiang et al., J. Vet. Diag. Invest., 12:381-84 (2000). As used herein, a vaccinated bird is an inoculated bird that is seropositive. A vaccinated bird may shed the attenuated, cold-adapted APV. APV shedding is typically detectable from about 5 days to about 7 days post inoculation, and may range from 3 days to 21 days post inoculation. Methods for detecting the shedding of an APV are known. Useful methods include the methods described in Shin et al., Arch. Virol., 145:1239-46(2000); and Goyal et al., J. Vet. Diagn. Invest., 12:166-68 (2000).

Vaccinated birds elicit an immunological response to a challenge with a virulent APV. Vaccinated birds can be resistant to or immune to a subsequent APV infection when challenged with a virulent form of APV. As a result, vaccinated birds that are subsequently challenged with a virulent APV may still pass slaughter inspections and continue to market. Methods and rating systems for passing or condemning birds destined for slaughter are known.

Virulent APV forms or isolates are those APV forms that cause a bird, which has not been exposed to APV and/or an APV vaccine, to develop severe clinical signs of an APV infection, to be unfit for market, and/or die. An APV form is virulent if it causes severe clinical signs of an APV infection in a bird at a dosage of at least 1.0×10² tissue culture infective dose (TCID₅₀).

Methods for computing virus titers are known. Any method for computing virus titers may be used. The TCID₅₀ is the reciprocal of the highest dilution of a virus that causes a specified reaction in 50% of the host cells inoculated with, or exposed to, that dilution of virus. It is common to express virus titers as TCID₅₀ when cell cultures are used as the indicator system. In such cases TCID₅₀ is the dilution that causes 50% of the cell cultures to elicit the specified reaction(s) and/or cytopathic effects, such as cell rounding. See, e.g., Cook et al., Avian Pathology, 18:511-522 (1989).

A vaccinated bird is resistant to or immune to an APV infection if it fails to develop severe clinical signs of APV infection after being challenged with a virulent APV. A resistant or immune bird may develop no clinical signs or mild clinical signs of an APV infection when exposed to a virulent APV. The clinical signs of APV and other upper respiratory infections in birds are known. The clinical signs of an APV infection in turkeys include profuse ocular and nasal discharge, watery eyes, unilateral or bilateral sinus swelling, facial edema or swelling, depression, coughing, sinusitis, airsacculitis, respiratory distress, and mortality. In laying turkeys, a drop in egg production associated with respiratory distress may be seen.

Immunologically effective dosages can be determined experimentally and may vary according to the type, size, age, and health of the bird vaccinated. For example, an effective amount for a two-week-old turkey poult may include an APV vaccine dosage of about 200 μl of a 1.0×10¹ TCID₅₀/ml stock vaccine solution. It is preferable to give a dosage of at least about 200 μl of a 1.0×10³ TCID₅₀/bird stock vaccine solution. Doses as high as 200 μl of a 1.0×10⁵ TCID₅₀/bird or a 1.0×10⁷ TCID₅₀/bird stock vaccine solution can be administered. Dosages smaller than 200 μl of a 1.0×10¹ TCID₅₀/bird stock solution may result in ineffective vaccinations, and dosages larger than 200 μl of a 1.0×10⁷ TCID₅₀/bird stock vaccine solution may be less cost effective. Older turkeys may require larger dosages. The vaccination may include a single inoculation or multiple inoculations. Other dosage schedules and amounts including vaccine booster dosages may be useful.

The age of the bird receiving a vaccination may depend upon the type of bird and the purpose for which the bird is being kept. For example, it may be preferable to inoculate meat-producing birds at a young age, perhaps as newborns or hatchlings or when the birds are only a few weeks old. Alternatively, it may be useful to vaccinate egg-producing birds at other times, e.g., shortly before they are about to lay (perhaps with a vaccine booster dosage) so that maternal antibodies may be transmitted to the young. Of course, it may also be useful to inoculate egg-laying birds at an early age to prevent APV infection in the egg-laying flock.

The immunologically effective dosage may be given to a bird using any known method for inoculating birds with attenuated, cold-adapted vaccines including direct application intranasally, intraocularly, and/or as a subcutaneous or intramuscular injection. The inoculation can be given to a single nostril or eye or divided between one or more nostrils or eyes. For example, a 200 μl dosage containing 1.0×10² TCID₅₀/ml can be evenly divided into four 50 μl dosages for both nostrils and eyes. The immunologically effective dosage may be given to a representative sample or subset of a flock. For example, at least 2 poults/1000 poults may be directly inoculated. Other bird samples including at least 1 bird/1000 birds, at least 5 birds/1000 birds, and at least 100 birds/1000 birds may be directly inoculated. The directly inoculated birds are then allowed to co-mingle with the rest of the flock and passively inoculate the other members of the flock. One way that the directly inoculated birds may inoculate other birds is through shedding of the attenuated, cold-adapted APV vaccine. Directly inoculating a subset of the flock creates a rolling or sequential vaccination as the attenuated, cold-adapted vaccine is passed from bird to bird. The number of vaccinated birds in the flock increases as the directly vaccinated birds interact with the rest of the flock. In the end, a majority or all of the birds should become vaccinated.

Alternatively, an immunologically effective dosage may be given to each member of a flock directly or the dosage can be applied to the food and/or water supply of a flock. For example, an immunologically effective dosage, e.g., about 1.0×10³ TCID₅₀/bird, can be aerosolized into the birds' habitat. Alternatively, an immunologically effective dosage can be dissolved in the water supply of a flock of birds. Most, if not all, of a flock should become vaccinated birds at approximately the same time when inoculating the flock through the food, water, or air supply. Dosages administered through the food or water supply can be easily computed by multiplying the amount a single bird eats or drinks per day by the number of birds to be inoculated to compute the unit of food or water consumed per day per bird. Then, the unit of food or water consumed per day is used to compute the vaccine dosage needed to dissolve in that unit of food or water so as to deliver at least 1.0×10² TCID₅₀/bird.

Attenuated, cold-adapted APV compositions or vaccines that are inactivated can be used in the same manner and under the same conditions used for attenuated, cold-adapted APV compositions. Inactivated APV vaccines are useful for any type of bird susceptible to APV infection including domesticated and wild birds. Methods for making inactivated virus vaccines are well known and include the use of formalin or β-propiolactone to make inactivated vaccines. Inactivated APV compositions or vaccines are typically administered by subcutaneous or intramuscular injection because they are no longer living. Such compositions may further include an adjuvant and/or pharmaceutical carrier.

Compositions containing an attenuated, cold-adapted APV also have uses other than as a vaccine. These compositions may be used to induce a bird to raise antibodies to APV to be used in diagnostic tests for identifying one or more APV isolates. Further the attenuated, cold-adapted APV may be used in a diagnostic assay for detecting the presence of anti-APV antibodies in the sera of a bird. Methods for raising and purifying antibodies are known. Methods for preparing diagnostic kits for detecting antibodies in a serum source are known.

Methods for Making Attenuated, Cold-Adapted Vaccines

Any APV form or isolate can be used to prepare an attenuated, cold-adapted APV vaccine. It may be preferable to use a virulent form of an APV. Suitable virulent APV isolates for preparing turkey vaccines include the European A, European B, Colorado, Minnesota 1A, Minnesota 1B, Minnesota 2A, Minnesota 2B, numerous other isolates that have been identified to date, and any other isolates that are identified in the future. Attenuated, cold-adapted APV vaccines may be combined with different vaccines or preventative methods directed to other avian diseases so as to produce birds that are relatively pathogen free, healthier, and/or resistant to more avian diseases than just APV. Other avian diseases include Ornithobacterium rhinotracheale, Bordetella avium, avian influenza, New Castle Disease, Mycoplasma spp., and Pasteurella multocida.

An attenuated, cold-adapted APV vaccine can be produced by first isolating an APV, propagating the APV at successively lower temperatures, and isolating an attenuated, cold-adapted APV vaccine. The attenuated, cold-adapted APV vaccines described herein can be produced using any known method for producing attenuated, cold-adapted vaccines. See, e.g. Williams et al., Avian Pathology, 20:585096 (1991). An illustrative method for producing an attenuated, cold-adapted APV vaccine is presented below.

APV can be isolated using any known method for isolating avian viruses. The methods described in Goyal et al., J. Vet. Diagn. Invest., 12:166-68 (2000) provide illustrative methods for isolating a turkey APV. The methods described in Goyal et al. (supra) may be adapted for other avian viruses. Known methods for isolating APV can include the step of obtaining specimens from birds, such as turkeys, that are exhibiting the clinical signs of an APV infection. Specimens can be obtained using tracheal swabs, turbinates, or by isolating the trachea and/or lungs. The specimens are then used to inoculate hosts such as embryonated chicken eggs (“ECE”), embryonated turkey eggs (“ETE”) or tracheal organ cultures (“TOC”).

Subsequently, the APV can be adapted to grow in other cell culture hosts such as chicken embryo fibroblasts (CEF), quail tumor cell lines or Vero cells using known methods. See Naylor et al., Vet. Bull., 63:439-49 (1993). Useful quail tumor cell lines include QT-35. Useful Vero cells include those available from the American Type Culture Collection (ATCC, 10801 University Blvd., Manassas, Va. 20110) having the designation CCL81. The inoculated cell culture hosts are incubated and serially passaged under standard conditions. It may be necessary for the inoculated cell culture hosts to undergo one or more blind passages before cytopathic effects, such as cell rounding, are observed. Cytopathic effects produced by various viruses in various cell cultures are well known. Cell cultures may be inoculated at each passage using any known method. Typically, cell cultures are subjected to repeated freezing and thawing to release the APV. Cellular debris is removed by centrifugation and the supernatant is then used to inoculate the next cell culture. Although the length of each passage may vary, each passage is typically 4-5 days in length. Identification of the passaged virus can be confirmed using any known method including RT-PCR. The presence of APV in the cell cultures during isolation can be confirmed using RT-PCR as described in Dar et al., Proc. Annual Meeting Am. Assoc. Vet. Lab. Diagn., 41:18 (1998).

Alternatively, APV may be directly isolated from an inoculation specimen using CEF, quail tumor cells, Vero cells, or other avian or non-avian cells. Briefly, tracheal swabs are used to inoculate monolayers of CEF, quail tumor cells, or Vero cells. The cell cultures are serially passaged until cytopathic effects are observed. APV particles can be isolated from cells in culture by freezing and thawing the cells. The experimental conditions and techniques used for serially passaging and isolating APV are known. Again, the presence of APV in the cell cultures during isolation can be confirmed using RT-PCR.

An isolated APV may be attenuated and made cold-adapted by serially passaging the APV in a suitable cell line at successively lower temperatures. Useful cell lines include CEF, quail tumor cells, and Vero cells. The number of passages needed to adequately attenuate APV can vary according to the APV strain and the type of cell culture used. Likewise, the number of times the temperature is reduced, the number of degrees of each temperature reduction, and the passage number(s) during which the temperature is reduced can depend upon the cells used to culture the APV and can depend upon the APV itself. As a virus becomes adapted to a cell line and/or a temperature, the length of each passage may shorten or the number of passages reduced. Accordingly, it may be necessary to test a putative vaccine at different passage levels so as to identify a properly attenuated and cold-adapted APV.

An APV is sufficiently attenuated when a dosage of about 1.0×10² TCID₅₀ to about 1.0×10⁶ TCID₅₀ causes no clinical signs or mild clinical signs of APV infection in a test bird. Mild clinical signs may include watery eyes, nasal discharge or cough in a flock from about 5 to about 12 days post inoculation (“dpi”). Continued passaging may further attenuate the APV. It may be necessary to serially passage the APV at least 10, 20, 30, 40, 50, 60, 70, 80, 90, 100, or more times to adequately attenuate the APV. Passaging the APV in cell culture can cause the virus to adapt to the cell culture causing it to become less virulent to its original host.

An APV is sufficiently cold-adapted when the APV grows at sub-optimal temperatures compared to a corresponding wild-type APV strain. Sub-optimal temperatures for APV include, for example, temperatures 2° C., 3° C., 4° C., 5° C., 6° C., 7° C., 8° C., 9° C., 10° C., 12° C., or 15° C., below the optimal growth temperature for the original APV strain. Reductions in temperature can be in 1° C. increments or in larger increments (e.g., 2° C., or 4° C. increments). The passage number at which the temperature is reduced also can vary depending, for example, on the cell culture. For example, the temperature at which the cells are grown can be reduced after 5, 10, 15, 20, 25, 30, 35, 40, 45, or 50 passages. As exemplified herein, cells or virus can be passaged for a particular number of passages (e.g., 41 passages at 37° C.) and then passaged a fewer number of times at each temperature reduction (e.g., 8 passages each at 35° C., 33° C., and 31° C.).

The attenuated, cold-adapted APV can be sequestered from cell culture using any known method for removing a virus from cell culture. Useful methods include repeated freezing and thawing or sonication of a cell culture used to passage the virus. An attenuated, cold-adapted APV is sequestered when it has been removed from its original host, serially passaged in cell culture a sufficient number times, and removed from cell culture so that administering a dosage of at least 1.0×10² TCID₅₀ of the attenuated, cold-adapted APV to a bird that has not been exposed to APV produces no clinical signs or mild clinical signs of an APV infection in the bird. An attenuated, cold-adapted APV is considered removed from culture if it is in a form that can be administered to a bird.

It may also be useful to isolate, identify, passage, and titrate an APV in a single cell type. For example, an APV vaccine may be developed using only CEF cells, only QT-35 cells, or only Vero cells. Furthermore, it may be useful to develop attenuated, cold-adapted APV vaccines to a variety of known APV isolates. Known APV isolates include European A, European B, Colorado, Minnesota 1A, Minnesota 1B, Minnesota 2A, and Minnesota 2B isolates. Varying and/or combining the APV isolate may enhance the immunogenic response in the vaccinated birds.

A useful attenuated, cold-adapted APV composition was deposited with the ATCC on Nov. 30, 2000 and received ATCC No. PTA-2751. To produce an attenuated, cold-adapted APV vaccine, APV was first serially passaged 7 times in CEF cells at 37° C. followed by 34 serial passages in Vero cells at 37° C. for a total of 41 passages. The APV was then passaged 8 times in Vero cells at 35° C., 8 times in Vero cells at 33° C., and 8 times in Vero cells at 31° C. These 65 passages caused the virus to adapt to the Vero cell culture, which caused it to become less virulent to its original host, in this case, turkeys. The attenuated, cold-adapted APV vaccine is an attenuated vaccine that is safe and immunogenic in turkeys.

Article of Manufacture Containing Isolated APV Polypeptides

Attenuated, cold-adapted APV compositions or vaccines as described herein can be combined with packaging materials including instructions for their use to be sold as articles of manufacture or kits. Components and methods for producing articles of manufacture are well known. The articles of manufacture may combine one or more attenuated, cold-adapted APV vaccines as described herein. In addition, the articles of manufacture may further include antibodies, indicator molecules, and/or useful agents for detecting other avian diseases together with attenuated, cold-adapted APV vaccines. Instructions describing how an attenuated, cold-adapted APV vaccine is effective for preventing the incidence of an APV infection, preventing the occurrence of the clinical signs of an APV infection, ameliorating the clinical signs of an APV infection, lowering the risk of the clinical signs of an APV infection, lowering the occurrence of the clinical signs of an APV infection and/or spread of APV infections in birds may be included in such kits. The article of manufacture can also include diagnostic molecules that are effective for detecting the presence of APV or other avian infections in birds.

It is to be understood that attenuated, cold-adapted APV vaccines themselves and/or articles of manufacture that include attenuated, cold-adapted APV vaccines can include other components conventional to the art, for example sterile water, pharmaceutical carriers, vaccine carriers, and buffers that are useful for maintaining the viability of the APV vaccines. The APV vaccines and/or articles of manufacture may also contain other attenuated and/or cold-adapted virus strains, microorganisms, and antigens that protect the inoculated birds against other avian diseases. Methods for producing such multi-effect vaccines are known. Conveniently the attenuated, cold-adapted APV vaccines may be provided in a pre-packaged form in quantities sufficient for a protective dose for a single bird or for a pre-specified number of birds in, for example, sealed ampoules, capsules or cartridges. Attenuated, cold-adapted APV vaccines may also be supplied in a freeze-dried form.

The invention will be further described in the following examples, which do not limit the scope of the invention described in the claims.

### EXAMPLES

### Example 1

Virus

Avian pneumovirus/MN/turkey/1-a/97 (APV/MN-1a) was isolated from an outbreak of respiratory illness in Minnesota turkeys by seven blind passages in chicken embryo fibroblasts (CEF). This virus was then passaged serially in Vero cells for 34 additional passages and was designated as p41 (7 passages in CEF and 34 passages in Vero cells).

### Example 2

Cells

The virus was propagated in monolayers of Vero-81 cells (ATCC-CCL81). The virus was inoculated in Vero cells and was allowed to absorb at 37° C. for 1 hr. Mock-infected Vero cells were kept as negative controls. After 1 hr, Eagle's minimum essential medium (MEM) with antibiotics (150 IU/ml penicillin, 150 μg/ml streptomycin, 50 μg/ml neomycin and 1 μg/ml Fungizone) and additives (Lactalbumin hydrolysate, Sigma, St. Louis, Mo.) were added to the flasks containing the infected Vero cells or the non-infected control Vero cells. The flasks were incubated at 37° C. in a humid atmosphere with 5% CO₂ until the characteristic cytopathic effects (CPE) of syncytium formation were observed. The virus was harvested from infected cells by three cycles of freezing and thawing at −70° C.

### Example 3

Cold-Adapted Virus

In an effort to adapt APV to grow at lower temperatures, the p41 virus was serially passaged at reduced incubation temperatures. The p41 virus was first incubated at 35° C. for 8 passages, followed by 8 passages at 33° C. and 8 additional passages at 31° C. The virus obtained after 8 passages at 31° C. was used as a cold-adapted strain and tested as a candidate APV vaccine.

### Example 4

Plaque Titration of Cold-Adapted Virus

The cold-adapted virus was titrated in 6-well plates by performing plaque assays using Vero cell monolayers as described (Pringle, In Virology: A Practical Approach, pp 95-117, IRL Press, Oxford, Washington, D.C.). Serial ten-fold dilutions of the cold adapted virus were made and each dilution was inoculated into duplicate wells containing Vero cells by adding 100 μl of each dilution. The plates were incubated in an incubator at 5% CO₂ for 1 hr at 31° C. After adsorption, minimal essential medium (MEM) with 2% fetal bovine serum, 1.5% noble agar and 100 μg/ml DEAE-dextran was added. After solidification of the top agar, plates were incubated at 31° C. for 5 days. The plaques in each dilution were counted after staining with 1% neutral red and the titer of the virus was expressed as plaque forming units per ml (pfu/ml).

### Example 5

Vaccination of Turkeys with Cold Adapted Virus

Eighty one-day-old turkey poults were obtained from a commercial hatchery and were reared in isolation. At two weeks of age, all birds were bled to determine the status of APV antibodies using an ELISA test (Chiang et al., 2000, J. Vet. Diag. Invest., 12:381-4). The poults were then divided into four groups designated A, B, C and D with 20 poults in each group. Two doses of the cold adapted virus were used in this study. The birds in groups A and B were inoculated with either 200 μl of a “high” dose (1.0×10^{4.3} TCID₅₀/ml) or 200 μl of a “low” dose (1.0×10^{2.3} TCID₅₀/ml) of the cold adapted virus. Each bird was inoculated by administering 50 μl of the virus into each eye and each nostril of the birds. Birds in groups C and D were inoculated with mock-infected Vero cells (50 μl into each eye and nostril). At four weeks of age (2 weeks post-vaccination), the birds in groups A, B, and C were challenged with an APV/MN-2a virus that had been passaged 13 times (7 passages in CEF cells and 6 passages in Vero cells). The titer of the APV/MN-2a virus used to challenge vaccinated and non-vaccinated birds was 1.0×10^{4.3} TCID₅₀/ml (Reed & Muench, 1938, Am. J Hyg., 27:493-7). The virus used to challenge vaccinated birds was originally isolated from nasal turbinates of 1-week-old turkeys and appeared to be more virulent than APV/MN-1a in preliminary studies. Each bird was challenged with 50 μl of virus in each eye and nostril (200 μl per bird). The birds were monitored daily for the appearance of clinical symptoms until the end of the study. Clinical scores were assigned according to the severity of clinical symptoms (Table 1). All birds were euthanized at 10 days post-challenge.

TABLE 1

| | Score assigned on the basis of: | |
|---|---|---|
| | Unilateral | Bilateral |
| Clinical symptoms | symptoms | symptoms |
| Nasal Discharge | 1 | 2 |
| Watery eyes | 2 | 3 |
| Moderate sinus swelling | 4 | 5 |
| Severe sinus swelling | 5 | 6 |

### Example 6

Serology and Virus Shedding

The birds were bled immediately before vaccination and then at weekly intervals after vaccination. APV antibody status was determined using ELISA (Chiang et al., 2000, J. Vet. Diag. Invest., 12:381-4). The titers were expressed as geometric mean titers for each experimental group (Goyal et al., 2000, Gobbles, 56:19).

At five days post-vaccination and at five days post-challenge, choanal swabs from each bird were collected in Eagle's MEM containing 1% fetal bovine serum and antibiotics (150 IU/ml penicillin, 150 μg/ml streptomycin, 50 μg/ml neomycin and 1 μg/ml fungizone). The swabs collected post-vaccination were combined into two pools of 10 swabs each from each of the four experimental groups (groups A, B, C, and D). The pooled samples were tested for the presence or absence of APV using reverse transcriptase polymerase chain reaction (RT-PCR; Shin et al., 2000, Arch. Virol., 145:1239-46). The swabs collected after the birds were challenged were examined individually for virus shedding using RT-PCR.

### Example 7

Effectiveness of Cold-Adapted Virus

The onset of CPE in Vero cells infected with the cold-adapted APV (cold-adapted to either 31° C. or 33° C.) was delayed compared to cells infected with a virus cultivated at higher temperatures (e.g., 37° C.). In general, complete CPE was observed after 5 days of incubation at 31° C. as opposed to 3 days at 37° C.

Following challenge, the non-vaccinated birds appeared initially depressed followed by development of clinical symptoms such as bilateral sinus swelling and bilateral nasal discharge. Clinical symptoms started appearing 5 days post-challenge with maximum symptoms observed 7-9 days post-challenge. A total of 18 birds out of 20 showed clinical symptoms of varying severity. The clinical score per bird in this group was 19.45/bird as compared to 0/bird in the vaccinated groups.

At 1 and 2 weeks post-vaccination, seroconversion was observed in birds from both vaccinated groups, although not all birds seroconverted. The birds in the non-vaccinated, non-challenged group remained negative for APV antibodies throughout the experiment, whereas birds in the non-vaccinated challenged group showed evidence of seroconversion 10 days post-challenge.

The pooled choanal swabs from vaccinated groups (groups A and B) were found to be positive by RT-PCR five days post-vaccination, whereas the birds in the mock-inoculated groups (groups C and D) were negative for virus shedding. Following challenge, choanal swabs were tested individually for virus shedding. None of the vaccinated, challenged birds in groups A and B were shedding the virus at 5 days post-challenge, whereas virus shedding was observed within days post-challenge in all non-vaccinated, challenged birds (group C). No virus shedding was observed in non-vaccinated, non-challenged birds.

What is claimed is:

1. A composition comprising an immunologically effective amount of an attenuated, cold-adapted avian pneumovirus (APV).

2. The composition of claim 1, further comprising an acceptable pharmaceutical carrier.

3. The composition of claim 1, wherein said attenuated, cold-adapted APV is adapted to grow at 31° C.

4. The composition of claim 1, wherein said composition is effective for lowering the risk of an APV infection in poultry.

5. The composition of claim 4, wherein said poultry is selected from the group consisting of turkeys and chickens.

6. The composition of claim 4, wherein said poultry is a turkey.

7. The composition of claim 1, wherein said composition is effective for ameliorating the clinical signs of an APV infection in a challenged bird.

8. A method for preparing an attenuated, cold-adapted avian pneumovirus (APV) composition comprising
a) inoculating a cell culture with an APV; and
b) serially propagating said infected cell culture at successively lower temperatures until said APV becomes attenuated and cold-adapted.

9. The method of claim 8, further comprising the step of removing said attenuated, cold-adapted APV from said infected cell culture.

10. The method of claim 8, wherein said cell culture is a non-avian cell culture.

11. The method of claim 8, wherein said APV is selected from the group consisting of the European A, European B, Colorado, Minnesota 1A, Minnesota 1B, Minnesota 2A, and Minnesota 2B isolates.

12. The method of claim 10, wherein said cell culture comprises Vero cells.

13. The method of claim 8, wherein said infected cell culture is serially propagated at least 20 times.

14. The method of claim 8, wherein said infected cell culture is serially propagated at least 40 times.

15. The method of claim 8, wherein said infected cell culture is serially propagated at least 60 times.

16. The method of claim 8, wherein said infected cell culture is serially propagated at least 100 times.

17. The method of claim 8, wherein said infected cell culture is serially propagated at 37° C.

18. The method of claim 8, wherein said infected cell culture is serially propagated at 35° C.

19. The method of claim 8, wherein said infected cell culture is serially propagated at 33° C.

20. The method of claim 8, wherein said infected cell culture is serially propagated at 31° C.

21. The method of claim 8, wherein said infected cell culture is serially propagated at 25° C.

22. The method of claim 8, wherein said infected cell culture is serially propagated 41 times at 37° C., 8 times at 35° C., 8 times at 33° C., and 8 times at 31° C.

23. The method of claim 8, wherein said attenuated, cold-adapted APV is effective for reducing the incidence of the clinical signs of an APV infection in poultry.

24. The method of claim 23, wherein said poultry is selected from the group consisting of turkeys and chickens.

25. The method of claim 23, wherein said poultry is a turkey.

26. A method for preparing an attenuated, cold-adapted avian pneumovirus (APV) composition, comprising the steps of:
a) inoculating an avian cell culture with an APV;
b) propagating said APV in said avian cell culture;
c) inoculating a non-avian cell culture with an APV isolated from said propagated avian cell culture;
d) serially propagating said non-avian infected cell culture at successively lower temperatures until said APV becomes attenuated and cold-adapted; and
e) isolating said attenuated, cold-adapted APV from said non-avian infected cell culture.

27. A method for reducing the risk of an APV infection in a bird comprising inoculating a bird with an immunologically effective amount of an attenuated, cold-adapted APV composition.

28. The method of claim 27, further comprising allowing said inoculated bird to become seropositive.

29. The method of claim 27, wherein said bird is inoculated with at least 1.0×10¹ TCID₅₀ of said attenuated, cold-adapted APV composition.

30. The method of claim 27, wherein said bird is inoculated with at least 1.0×10² TCID₅₀ of said attenuated, cold-adapted APV composition.

31. The method of claim 27, wherein said bird is inoculated with at least 1.0×10³ TCID₅₀ of said attenuated, cold-adapted APV composition.

32. The method of claim 27, wherein said bird is inoculated with at least 1.0×10⁵ TCID₅₀ of said attenuated, cold-adapted APV composition.

33. The method of claim 27, wherein said bird is inoculated with at least 1.0×10⁷ TCID₅₀ of said attenuated, cold-adapted APV composition.

34. The method of claim 27, wherein said attenuated, cold-adapted APV composition is applied to an eye of said bird.

35. The method of claim 27, wherein said attenuated, cold-adapted APV composition is applied to a nostril of said bird.

36. The method of claim 27, wherein said attenuated, cold-adapted APV composition is supplied in the drinking water of said bird.

37. The method of claim 27, wherein said attenuated, cold-adapted APV composition is aerosolized.

38. The method of claim 27, wherein said bird is a member of a flock of birds and wherein said bird causes a majority of said flock to become seropositive.

39. The method of claim 27, wherein said method is effective for reducing the incidence of the clinical signs of an APV infection in a challenged bird.

40. The method of claim 27, wherein said attenuated, cold-adapted APV in said composition is adapted to grow at 31° C.

41. An inoculated bird, wherein said bird contains an inoculant of an immunologically effective amount of an attenuated, cold-adapted APV.

42. The bird of claim 41, wherein said bird is seropositive for APV.

43. The bird of claim 41, wherein said bird is a turkey.

44. A body part of the turkey of claim 41.

45. The body part of claim 44, wherein said body part comprises a meat portion.
